(19) United States
(12) Patent Application Publication
(10) Pub. No.: US 2014/0371550 A1
(43) Pub. Date: Dec. 18, 2014

(54) ELECTROLYTIC BIOSENSOR

(71) Applicants: Justin Lyle Hoffman, San Antonio, TX (US); Michael John Horwath, Boerne, TX (US); Karin Haumann, San Antonio, TX (US); Jennifer Stephanie Schmidt, Lytle, TX (US); Daniel Thomas DeArmond, San Antonio, TX (US)

(72) Inventors: Justin Lyle Hoffman, San Antonio, TX (US); Michael John Horwath, Boerne, TX (US); Karin Haumann, San Antonio, TX (US); Jennifer Stephanie Schmidt, Lytle, TX (US); Daniel Thomas DeArmond, San Antonio, TX (US)

(73) Assignee: Board of Regents of The University of Texas System, Austin, TX (US)

(21) Appl. No.: 14/113,347

(22) PCT Filed: Apr. 19, 2012
(86) PCT No.: PCT/US12/34306

§ 371 Date: Jul. 25, 2014

Related U.S. Application Data
(60) Provisional application No. 61/478,442, filed on Apr. 22, 2011.

(51) Int. Cl. A61B 5/02; A61B 5/00; A61B 5/1473; A61M 27/00; A61B 5/07

(52) U.S. Cl. CPC A61B 5/02042; A61M 27/00; A61B 5/076; A61B 5/1473; A61B 5/4848; A61B 5/686; A61B 5/42
USPC 600/302

(57) ABSTRACT

An electrolytic biosensor for use in intracorporeal leak detection may be incorporated into a surgical drain that is installed post-operatively at the site of a wound. The electrolytic biosensor may be used to monitor drain effluent flowing through the surgical drain by measuring a fluid conductivity of the drain effluent. Certain changes, patterns, or responses of the so measured fluid conductivity values may be indicative of a physiological condition of the wound. For example, when the wound is a gastrointestinal anastomosis, the fluid conductivity values measured with the electrolytic biosensor may be used to characterize a fluid integrity of the gastrointestinal anastomosis. An electrolytic biosensor system may include an application that records conductivity data and provides notifications, such as a patient alarm, when a leak is detected.

### CROSS REFERENCE TO RELATED APPLICATIONS

This application is a national stage of PCT/US2012/034306, filed on Apr. 19, 2012, which claims priority from U.S. Provisional Patent Application No. 61/478,442, filed on Apr. 22, 2011, and entitled “ELECTROLYTIC BIOSENSOR.” Both of these applications are herein incorporated by reference in their entirety.

### BACKGROUND

1. Field of the Disclosure

This disclosure relates to the field of electrolytic biosensors, and more particularly to a measuring unit for electrolytic intracorporeal testing.

2. Description of the Related Art

After internal surgery, a surgical drain may be installed to drain effluent from a wound site. The surgical drain may be a medical device that provides a drainage path away from the wound area.

For example, surgery involving the gastrointestinal tract is commonly performed for a variety of reasons. In many cases, gastrointestinal surgery involves the division of the gastrointestinal tract and removal of a segment of the gastrointestinal tract. When the gastrointestinal tract is divided and/or a segment of the gastrointestinal tract is removed, a subsequent re-connection is performed to restore gastrointestinal continuity using suture material, surgical stapling devices, and/or various reinforcing materials. This re-connection is referred to as a gastrointestinal anastomosis.

After gastrointestinal anastomosis operations, leakage of material from the gastrointestinal tract into surrounding tissues or body cavities through the gastrointestinal anastomosis may occur. Leakage may result in significant patient morbidity and mortality due to the fact that gastrointestinal contents generally contain bacteria while the body compartments surrounding the gastrointestinal tract are generally sterile and ill equipped to mount an appropriate immunologic defense against bacterial elements. Ultimately, the tissues of the gastrointestinal tract in the area of an anastomosis undergo regenerative processes that completely seal any areas of potential leakage. In the first few weeks after surgery, while these regenerative processes are occurring, the surgical procedure used to form the anastomosis is expected to establish enough temporary integrity of the gastrointestinal wall to prevent leakage of materials from the gastrointestinal tract into surrounding body compartments. Despite the efforts of surgeons, anastomotic leaks may occur while the tissues undergo regeneration during the first few weeks after surgery. When a surgical drain is in place, leakage of material through the gastrointestinal anastomosis may be evident in the surgical drain.

In U.S. Pat. No. 7,899,508, a monitoring device for intracorporeal leaks is disclosed. The disclosed monitoring device may include sensors and a measurement unit producing an output signal indicative of an impedance near a wound, such as a gastrointestinal anastomosis. A measurement unit producing an output signal indicative of impedance may be difficult to interpret accurately and may be poorly integrated into a modern post-operative instrumentation environment, which may result in an insurmountable barrier to widespread application.

### BRIEF DESCRIPTION OF THE DRAWINGS

FIG. 1 is a block diagram of selected elements of an embodiment of an electrolytic biosensor system;

FIG. 2 is a block diagram of selected elements of an embodiment of an electrolytic biosensor system;

FIG. 3 is a block diagram of selected elements of an embodiment of an electrolytic biosensor system;

FIG. 4 is a block diagram of selected elements of an embodiment of an electrolytic biosensor system;

FIG. 5 illustrates an embodiment of a method for performing measurements using an electrolytic biosensor; and

FIG. 6 illustrates an embodiment of a method for determining wound integrity using an electrolytic biosensor.

### DESCRIPTION OF THE EMBODIMENT(S)

The present disclosure pertains to a novel electrolytic biosensor for use in intracorporeal leak detection. As will be described in detail herein, the disclosed electrolytic biosensor may be incorporated into a surgical drain that is installed post-operatively at the site of a wound. The electrolytic biosensor may be used to monitor drain effluent flowing through the surgical drain by measuring a fluid conductivity of the drain effluent. Certain changes, patterns, or responses of the so measured fluid conductivity values may be indicative of a physiological condition of the wound. For example, when the wound is a gastrointestinal anastomosis, the fluid conductivity values measured with the electrolytic biosensor may be used to characterize a fluid integrity of the gastrointestinal anastomosis, as will be described in further detail below. In this manner, reliable and early warning of dangerous leaks in the gastrointestinal anastomosis may be provided, which may significantly contribute to reduced complications and morbidity.

Another advantage of the electrolytic biosensor disclosed herein may be an economic benefit for medical facilities who purchase and offer medical devices for treatment of patients. Embodiments of the disclosed electrolytic biosensor may be relatively inexpensive compared to conventional capital equipment used to carry out data collection and/or analysis. For example, an ability of the electrolytic biosensor disclosed herein to transmit patient data and related messages using existing network infrastructure and/or to mobile user devices may enable simplified integration for users and may provide significant cost-to-benefit improvements over conventional methods for a medical facility.

In one aspect, an electrolytic biosensor device may include a processor configured to access memory media and a wireless interface. The memory media may store processor instructions executable by the processor. The processor instructions may be executable to balance a bridge circuit that includes a probe installed in a surgical drain at an intracorporeal wound site. The processor instructions may further be executable to acquire conductivity values from the bridge circuit corresponding to conductivity of a drain effluent in contact with the probe when flowing through the surgical drain. The electrolytic biosensor device may then store the conductivity values and/or transmit the conductivity values via the wireless interface.

In certain embodiments, the electrolytic biosensor device may be incorporated into the surgical drain. The electrolytic biosensor device may include the bridge circuit, a sensor interface coupling the bridge circuit to the probe, and a power source configured to drive the sensor interface. The electrolytic biosensor device may also include a power control module configured to switch connections to the sensor interface. The processor instructions executable to acquire the conductivity values may include processor instructions executable to perform digital signal processing on output signals from the bridge circuit.

In various embodiments, the electrolytic biosensor device may include processor instructions executable to determine wound integrity based on the conductivity values and transmit an indication of the wound integrity via the wireless interface.

In another aspect, a method of detecting wound integrity using an electrolytic biosensor may include determining an expected ingestion profile for a patient ingesting a trace fluid, including estimating a conductivity change over time at a gastrointestinal wound site of the patient. Responsive to receiving an indication of the ingesting of the trace fluid, the method may further include collecting ingestion profile conductivity data using an electrolytic probe at the gastrointestinal wound site of the patient. The electrolytic probe may be installed in a surgical drain and may be configured to measure conductivity of an effluent fluid flowing from the gastrointestinal wound site through the surgical drain. The method may still further include analyzing the ingestion profile conductivity data to determine fluid integrity of the gastrointestinal wound site, including comparing the ingestion profile conductivity data with the expected ingestion profile.

In some embodiments, the trace fluid may have a substantially higher conductivity than a nominal conductivity value of the effluent fluid, while the conductivity change may be a substantial increase in conductivity. The method operation of determining the expected ingestion profile may include using a physiological model describing specific attributes of the patient and estimating a degree of leakage at the gastrointestinal wound site. The method operation of collecting ingestion profile conductivity data may include storing ingestion profile conductivity data, while the method operation of analyzing the ingestion profile conductivity data may include comparing new ingestion profile conductivity data to previously stored ingestion profile conductivity data.

In particular embodiments, the method may further include wirelessly transmitting an indication of the fluid integrity of the gastrointestinal wound site to a wireless user device. The method may also include transmitting an indication of the fluid integrity of the gastrointestinal wound site to a server via a network. The method operation of analyzing the ingestion profile conductivity data to determine fluid integrity of the gastrointestinal wound site may include determining a degree of leakage at the gastrointestinal wound site. Based on the degree of leakage, the method may include generating an alarm associated with the patient.

In yet another aspect, a non-transitory computer readable memory media may store processor instructions executable by a processor to operate an electrolytic biosensor device. The processor instructions may include instructions executable to balance a bridge circuit that includes an electrolytic probe installed in a surgical drain at an intracorporeal wound site of a patient. The electrolytic probe may include a conductivity cell for measuring conductivity. The processor instructions may be executable to acquire conductivity data from the bridge circuit corresponding to conductivity of a drain effluent flowing through the conductivity cell and determine fluid integrity of the intracorporeal wound site by comparing the conductivity data with previously-stored reference conductivity data. The processor instructions may further be executable to transmit an indication of the fluid integrity of the intracorporeal wound site.

In certain embodiments, the processor instructions executable to acquire the conductivity data may include processor instructions executable to control a sensor interface to the conductivity cell and process an output signal from the conductivity cell to generate the conductivity data. Based on the indication of the fluid integrity of the intracorporeal wound site, the processor instructions may further be executable to generate an alarm associated with the patient. The processor instructions executable to transmit an indication of the fluid integrity may include processor instructions executable to send a wireless message to a wireless user device. The memory media may further include processor instructions executable to transmit the conductivity data for the patient to a server. The processor instructions executable to compare the conductivity data with previously-stored reference conductivity data may include processor instructions executable to retrieve reference conductivity data for the patient.

In the following description, details are set forth by way of example to facilitate discussion of the disclosed subject matter. It should be apparent to a person of ordinary skill in the field, however, that the disclosed embodiments are exemplary and not exhaustive of all possible embodiments.

Throughout this disclosure, a hyphenated form of a reference numeral refers to a specific instance of an element and the un-hyphenated form of the reference numeral refers to the element generically or collectively. Thus, for example, widget 12-1 refers to an instance of a widget class, which may be referred to collectively as widgets 12 and any one of which may be referred to generically as a widget 12.

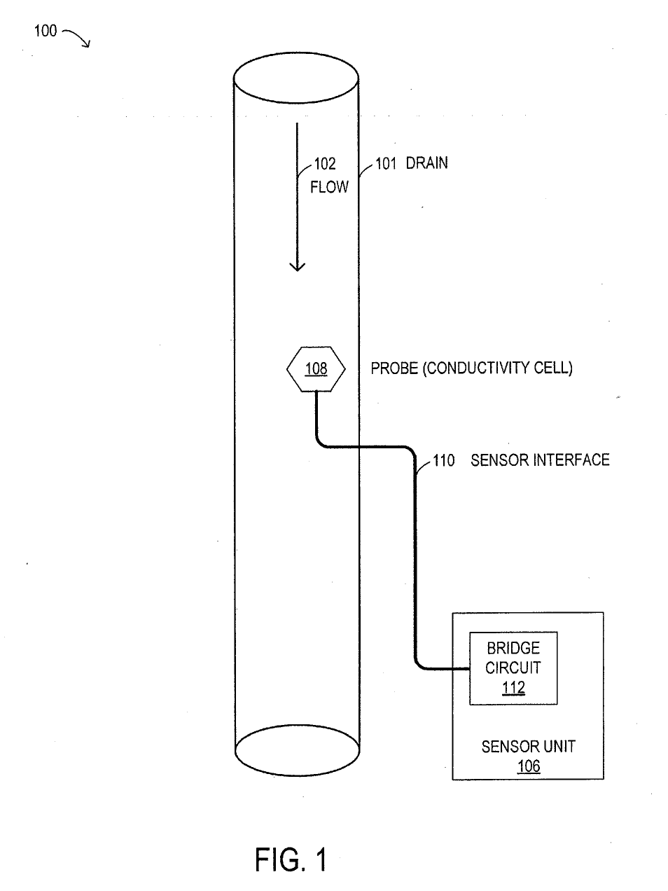

Turning now to the figures, FIG. 1 shows a block diagram of selected elements of an embodiment of electrolytic biosensor system 100. Electrolytic biosensor system 100 is shown with respect to surgical drain 101 through which a drain effluent (not shown) flows in direction 102. Surgical drain 101 may represent a portion of any of a variety of devices, such as a Jackson Pratt drain, a fenestrated tube, or similar device. Surgical drain 101 may be subject to pressure or vacuum to assist in flow transport of the drain effluent. In certain embodiments, surgical drain 101 may represent a disposable medical device.

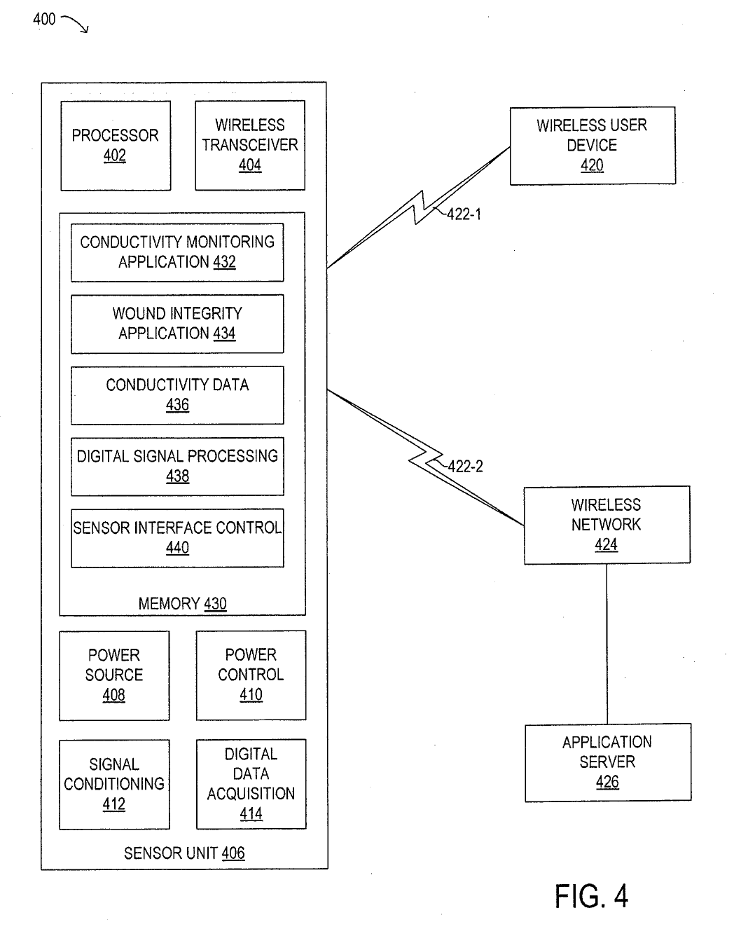

As shown in FIG. 1, installed within surgical drain 101 is conductivity probe 108, which may be configured with electrodes (not shown) that are configured to come in physical contact with the drain effluent flowing through surgical drain 101. The electrodes may form a conductivity cell for measuring conductivity of a material coming in physical contact therewith, such as the drain effluent. Conductivity probe 108 may be coupled to sensor unit 106 via sensor interface 110. Also shown included with sensor unit 106 is bridge circuit 112, which may provide signal conditioning and associated power connections to conductivity probe 108 via sensor interface 110 (see also FIG. 4). Bridge circuit 112 may be employed to improve sensitivity and/or accuracy of conductivity measurements performed using electrolytic biosensor system 100. It is noted that in various embodiments, at least a portion of bridge circuit 112 may be implemented in a separate device, and/or incorporated within conductivity probe 108, and/or included with sensor interface 110.

In the exemplary configuration of electrolytic biosensor system 100, sensor unit 106 may reside external to surgical drain 101. In certain embodiments, surgical drain 101 represents a distal portion, while a proximal portion of surgical drain 101 may be installed at an intracorporeal wound site. In other embodiments, surgical drain 101 represents a proximal end near the wound site, while sensor interface 110 may reside within surgical drain 101 for an extended distance. Conductivity probe 108 may be formed in various sizes and configurations and may be affixed or integrated within a portion of surgical drain 101. Conductivity probe 108 may include exposed portions of electrodes (not shown) in contact with the drain effluent and which have individual galvanic connections to sensor unit 106 via sensor interface 110, as will be described in further detail. Thus, certain portions of conductivity probe 108 may be formed with biocompatible electrode materials, including noble metals, metal alloys, conductive polymer over metal, cermets, nanotubes, nanoparticles, nanomaterials, or various combinations thereof. Examples of noble metals and alloys that are considered biocompatible include stainless steel, Co—Cr, Ti, Ta, Ir, and Pt, among others. An example of a biocompatible polymer coated conductor is polypyrrole-coated indium tin oxide. An example of a biocompatible conductive polymer suitable for coating metal is poly(3,4-ethylenedioxythiophene) poly(styrenesulfonate) also referred to as PEDOT. An example of a biocompatible nanotube includes carbon nanotubes.

In operation of electrolytic biosensor system 100, as the drain effluent passes over (or through) electrodes of conductivity probe 108, sensor unit 106 may provide electrical stimulation across a pair of the electrodes, while simultaneously sampling an output signal to generate conductivity values. The electrical stimulation and the output signal may be connected using sensor interface 110. As shown in FIG. 1, sensor unit 106 includes bridge circuit 112, of which one branch may be an electrode pair included in conductivity probe 108. In one embodiment (not shown), bridge circuit 112 may be included in conductivity probe 108 and sensor interface 110 may provide two, three or four wire connection to bridge circuit 112. In another embodiment (not shown), a voltage or current divider may be used to measure the conductivity of the fluid using connections provided by sensor interface 110. The electrical stimulation for the electrodes and/or the bridge circuit may be in the form of direct current (DC) or alternating current (AC), in various embodiments. The output signal provided by the bridge circuit configuration may be acquired and digitized by sensor unit 106 (see also FIG. 4). Sensor unit 106 may further be configured to intelligently monitor conductivity values so acquired and perform analysis routines on the conductivity values. Sensor unit 106 may also be configured to communicate externally to transmit the conductivity values and/or the results of the analysis routines, which may include a confirmation of wound integrity, or a notification (e.g., a patient alarm) that a leak through the wound has been detected. In certain embodiments, sensor unit 106 may be removably coupled at sensor interface 110 and may initiate operation when plugged in. Sensor unit 106 may further be equipped with a computer bus interface (not shown in FIG. 1), such as a Universal Serial Bus (USB), to which a computer processor or a USB device may be coupled to effect measurement, testing, and/or download of conductivity data.

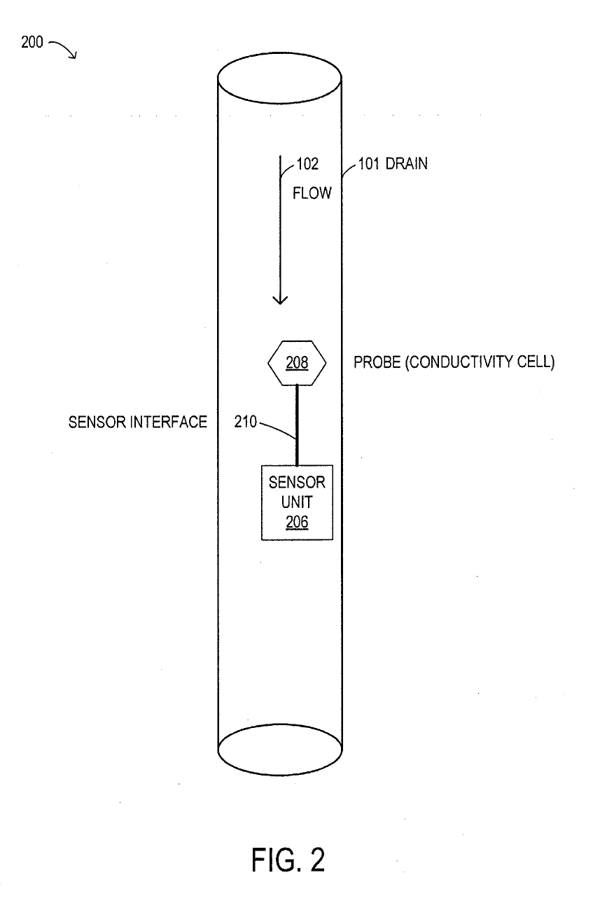

Turning now to FIG. 2, a block diagram of selected elements of an embodiment of electrolytic biosensor system 200 is shown. It is noted that like numbered elements in FIG. 2 correspond to the same elements in FIG. 1, such as surgical drain 101 and direction 102 of the drain effluent (not shown). In electrolytic biosensor system 200, conductivity probe 208 may be substantially similar to conductivity probe 108 (see FIG. 1), while sensor unit 206 may provide substantially similar functionality as described above with respect to sensor unit 106. In electrolytic biosensor system 200, sensor unit 206 is now integrated within surgical drain 101. Sensor unit 206 may represent a microdevice, such as a miniaturized circuit board and/or an integrated semiconductor circuit device. Sensor interface 210 may embody substantially similar connectivity as described above with respect to sensor interface 110, while being integrated into a portion of surgical drain 101. In certain embodiments, sensor interface 210 may be implemented as an ultra-thin laminate that is attached to an interior surface of surgical drain 101. In one embodiment, sensor interface 210 may enable conductivity probe 208 to reside at a proximate portion of surgical drain 101, while sensor unit 206 resides at a distal portion. It is noted that at least some portions of sensor unit 206, sensor interface 210, and conductivity probe 208 may be packaged to be thermally, electronically, and/or chemically isolated from an environment within surgical drain 101.

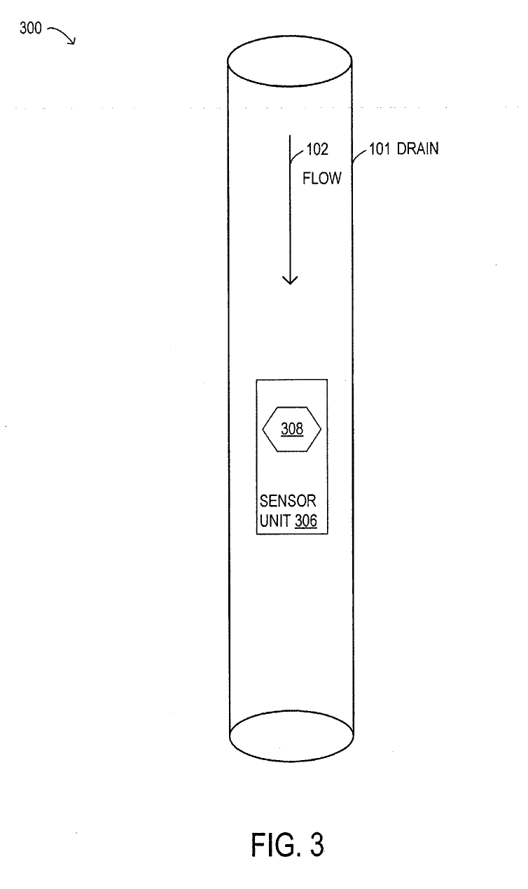

Turning now to FIG. 3, a block diagram of selected elements of an embodiment of electrolytic biosensor system 300 is shown. It is noted that like numbered elements in FIG. 3 correspond to the same elements in FIGS. 1 and 2, such as surgical drain 101 and direction 102 of the drain effluent (not shown). In electrolytic biosensor system 200, conductivity probe 308 may be substantially similar to conductivity probe 108 (see FIG. 1), while sensor unit 306 may include substantially similar functionality as described above with respect to sensor unit 106. In electrolytic biosensor system 300, conductivity probe 308 is now integrated within sensor unit 306 inside surgical drain 101 as a single device that incorporates sensor interfaces (not explicitly shown in FIG. 3). Sensor unit 306 may represent a microdevice, such as a miniaturized circuit board and/or an integrated semiconductor circuit device. Sensor unit 306 may be placed at any desired location within surgical drain 101. In certain embodiments, sensor unit 306 may be implemented as a microdevice that is attached to, or integrated within, a portion of surgical drain 101. It is noted that at least some portions of sensor unit 306 and conductivity probe 308 may be packaged to be thermally, electronically, and/or chemically isolated from an environment within surgical drain 101. It is further noted that surgical drain 101 may include or incorporate additional connections (not shown), such as a power connection, between sensor unit 306 and an external system (not shown).

Referring now to FIG. 4, a block diagram of selected elements of an embodiment of an electrolytic biosensor system 400 is depicted. In electrolytic biosensor system 400, sensor unit 406 may represent various embodiments, including sensor units 106, 206, and 306, and is shown without a conductivity probe for descriptive clarity. Also omitted from FIG. 4 is surgical drain 101 (see FIGS. 1-3).

As shown in FIG. 4, sensor unit 406, which may be a microdevice or a miniaturized device, includes numerous elements, and may include additional elements (not shown in FIG. 4) in various embodiments. Sensor unit 406 is shown including processor 402, wireless transceiver 404, memory 430, power source 408, power control 410, signal conditioning 412, and digital data acquisition 414. Memory 430 is depicted in FIG. 4 including conductivity monitoring application 432, wound integrity application 434, conductivity data 436, digital signal processing 438, and sensor interface control 440. Accordingly, sensor unit 406 may comprise elements configured to function as an embodiment of an electronic device capable of executing program instructions. Sensor unit 406 may further include at least one shared bus (not shown in FIG. 4) for interconnectivity among internal elements, such as those depicted in FIG. 4, as well as connectivity to sensor interfaces, such as sensor interface 110, 210, and 310 (not shown in FIG. 4, see FIGS. 1-3).

Processor 402 may represent at least one processing unit and may further include internal memory, such as a cache for storing processor executable instructions. In certain embodiments, processor 402 serves as a main controller for sensor unit 406. In various embodiments, processor 402 is operable to perform operations associated with electrolytic biosensor systems, as described herein. Processor 402 may access memory 430 to receive executable instructions and/or to store data in memory 430.

In FIG. 4, wireless transceiver 404 may represent a communications transceiver providing an interface for any of a number of communication links. In certain embodiments, wireless transceiver 404 supports wireless communication links, such as infrared (IR), radio frequency (RF), and audio, among others. Examples of RF wireless links include the IEEE 802.xx family, such as WiFi® (IEEE 802.11) and Bluetooth® (IEEE 802.15.1). In addition to wireless transceiver 404, sensor unit 406 may further support mechanically connected communication links, such as galvanically wired connections, sensor interface connections, connections to external antennas, network connections (i.e., Ethernet), etc., and may accordingly include a physical adapter or receptacle (not shown in FIG. 4) for receiving such connections. Wireless transceiver 404 may transform an instruction received from processor 402 into a signal sent via wireless communication link 422. It is noted that wireless transceiver 404 may be a bidirectional interface, such that responses, such as commands, information, or acknowledgements, may be received via wireless communication link 422.

In FIG. 4, wireless user device 420 may represent a smart phone or other mobile communication device with application processing capacity. Wireless user device 420 may be in possession of a physician or other medical staff charged with the care of a patient whose post-operative wound is being monitored by electrolytic biosensor system 400. It is noted that an application executing on wireless user device 420 may specifically be configured to operate with one or more instances of sensor unit 406. In one embodiment, a message may be sent to wireless user device 420 via wireless communication link 422-1 and an acknowledgement of the message may be received from wireless user device 420 via wireless communication link 422-1, such that the message is sent and the acknowledgment is received via wireless transceiver 404. Wireless transceiver 404 may further represent a client device in wireless network 424 that is accessible via wireless communication link 422-2. Wireless network 424 may be a wide-area wireless network, such as a cellular telephony network, for example. Wireless network 424 may enable sensor unit 406 to communicate with application server 426 to exchange application data, commands, and measurement data, as desired.

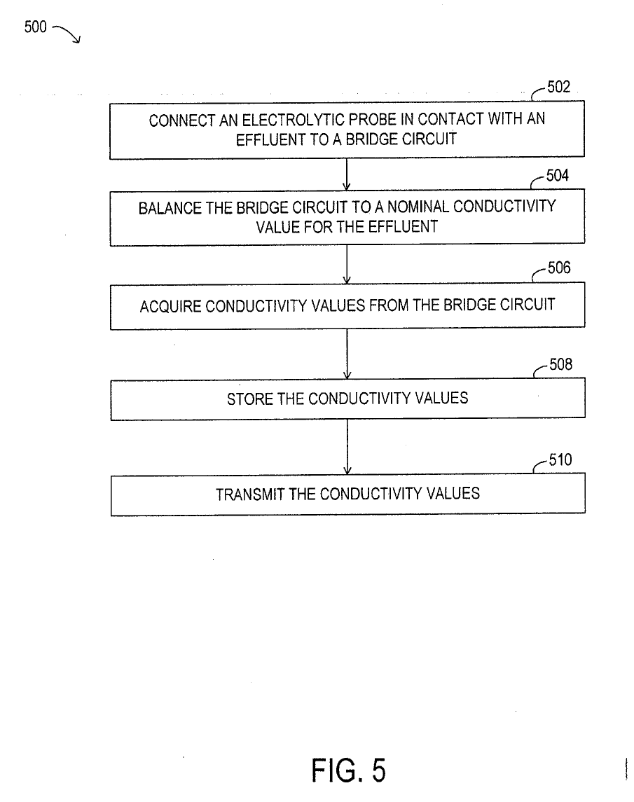

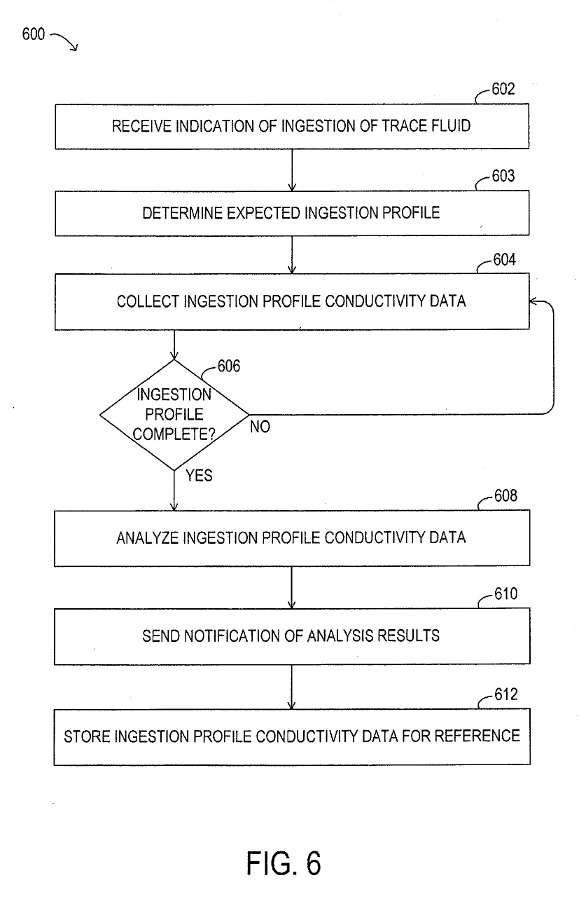

In FIG. 4, memory 430 encompasses persistent and volatile media, fixed and removable media, magnetic and semiconductor media, or a combination thereof. Memory 430 is operable to store instructions, data, or both. Memory 430 as shown includes data, which may be in the form of sets or sequences of executable instructions, namely, conductivity monitoring application 432, wound integrity application 434, digital signal processing 438, and sensor interface control 440. Conductivity monitoring application 432 may include processor executable instructions to measure conductivity values from a conductivity probe coupled to (or integrated with) sensor unit 406 (see also FIG. 5). Wound integrity application 434 may include processor executable instructions to determine whether or not a wound for which conductivity values are measured is leaking (see also FIG. 6). Digital signal processing 438 may include processor executable instructions to process output signals from a conductivity probe (not shown in FIG. 4, see FIGS. 1-3), including operations such as digital filtering, normalization, scaling, numerical calculations, differential calculations, etc. In certain instances, digital signal processing 438 may perform operations in substantially real-time or immediately upon measurement. Sensor interface control 440 may include processor executable instructions to control a sensor interface (not shown in FIG. 4, see FIGS. 1-2), including switching or power and output signals, as well as other logical operations. During operation of sensor unit 406, sensor interface control 440 may accordingly operate in conjunction with power source 408, power control 410, signal conditioning 412, and digital data acquisition 414. Memory 430 is further shown including conductivity data 436, representing conductivity values that have been acquired, such as conductivity data reference values and/or ingestion profile conductivity data, for example.

Also shown included with sensor unit 406 in FIG. 4 is power source 408, which may represent a local power source, such as a battery and/or an interface to an external power supply. Power source 408 may be configured for DC, AC or both, and may be configured to convert between various levels of AC and/or DC power. Power source 408 may be configured to regulate an output voltage or an output current, as desired. Power control 410 may represent a switching system for routing power to desired interfaces, such as via sensor interfaces (not shown in FIG. 4, see FIGS. 1-3). Power control 410 may be configured to route and switch power connections on command or in a pre-programmed manner, such as under control of processor 402 and/or sensor interface control 440. Signal conditioning 412 may represent hardware components for isolating and/or amplifying output measurement signals, such as from conductivity probes via sensor interfaces. It is noted that signal conditioning 412 may include filtering capability, in particular, for selectively isolating a desired bandwidth of conductivity signals. It is further noted that certain signal conditioning operations may be performed by signal conditioning 412 and/or by digital signal processing 438, as desired. In certain embodiments, a bridge circuit (see also bridge circuit 112 in FIG. 1) and/or a voltage divider may be implemented using signal conditioning 412. Digital data acquisition 414 may represent any of a number of different types of analog-to-digital converters (ADC), such as, but not limited to, successive approximation ADCs, flash ADCs, Sigma-Delta ADCs, integrating ADCs, ramp ADCs, and digital multimeters (DMM), among others.

Turning now to FIG. 5, an embodiment of novel method 500 for performing measurements using an electrolytic biosensor is illustrated in flow chart form. In one embodiment, method 500 is performed by conductivity monitoring application 432 (see FIG. 4). It is noted that certain operations described in method 500 may be optional or may be rearranged in different embodiments. Although method 500 is described with respect to a bridge circuit, in some embodiments, another type of signal conditioning arrangement for conductivity measurements may be implemented.

Method 500 may begin by connecting (operation 502) an electrolytic probe in contact with an effluent to a bridge circuit. In various embodiments, the electrolytic probe is included in conductivity probe 108, 208, 308 (see FIGS. 1-3). The electrolytic probe may be a conductivity cell with electrodes configured to probe conductivity (or resistance or impedance) from a drain effluent flowing through a surgical drain installed at an intracorporeal wound site. The connection in operation 502 may be performed by power control 410 and/or sensor interface control 440 (see FIG. 4). The bridge circuit may also be included in conductivity probe 108, 208, 308 (see FIGS. 1-3). The bridge circuit may be balanced (operation 504) to a nominal conductivity value for the effluent. The balancing of the bridge circuit may correspond with a background, or baseline, level of conductivity in the electrolytic probe. The balancing may be performed digitally by processor 402 in conjunction with digital data acquisition 414 and/or sensor interface control 440 (see FIG. 4). The balancing may be performed using AC and/or DC excitation of the bridge circuit. Conductivity values may be acquired (operation 506) from the bridge circuit. The acquisition in operation 506 may be performed with digital data acquisition 414 (see FIG. 4). Operation 506 may continue with acquisition of conductivity values at a given interval over a desired period of time. The acquired conductivity values may be processed, such as by averaging or digital filtering using digital signal processing 438, to improve a signal to noise ratio of the conductivity values. The conductivity values may be calibrated against a standard sample and acquired in normalized form. In certain embodiments, high frequency conductivity values may be acquired in operation 506. The conductivity values may be stored (operation 508). The conductivity values may be stored on sensor unit 406 or on application server 426 (see FIG. 4). The conductivity values may be transmitted (operation 510). The conductivity values may be transmitted to wireless user device 420 and/or application server 426. It is noted that application server 426 may be configured to store and receive conductivity values from a plurality of sensor units. Application server 426 may be configured to store conductivity values in a database (not shown). It is noted that operations 508 and 510 may also be implemented using a local interface, such as with a removable USB storage device on which the conductivity values are stored.

Turning now to FIG. 6, an embodiment of novel method 600 for determining wound integrity using an electrolytic biosensor is illustrated in flow chart form. In one embodiment, method 600 is performed by wound integrity application 434 executing on sensor unit 406 (see FIG. 4) for testing the integrity of a gastrointestinal anastomosis. In other embodiments, wound integrity application 434 may be configured to perform method 600 by execution on application server 426, in conjunction with conductivity monitoring application 432, for example, by transmitting conductivity data to application server 426. It is noted that certain operations described in method 600 may be optional or may be rearranged in different embodiments. Method 600 may be performed using any combination of electrolytic biosensor systems 100, 200, 300 to determine the integrity of a post-operative intracorporeal wound, such as a gastrointestinal anastomosis. It is noted that method 600 may be performed intermittently or continuously for wound condition monitoring and tracking. It is further noted that method 600 may be used to monitor wound integrity in substantially real time and to generate notifications of wound integrity in real time, such as a patient alarm.

Method 600 may begin by receiving (operation 602) an indication of ingestion of a trace fluid. The indication may be received by wireless user device 420 (see FIG. 4) or application server 426. The indication may include a time of ingestion, an amount of ingested trace fluid, an identity of a patient ingesting the trace fluid, and/or an identifier for the trace fluid. In certain embodiments, the identifier for the trace fluid is a conductivity of the trace fluid. Then, an expected ingestion profile may be determined (operation 603). The expected ingestion profile may reflect an expected conductivity change over time at a point in the gastrointestinal tract where a wound, whose integrity is being determined by method 600, is located. The expected ingestion profile may be determined using reference ingestion profile conductivity data stored in operation 612. In certain embodiments, the expected ingestion profile may be determined using physiological models with input parameters describing the patient, such as gender, age, height, weight, metabolism values, etc. The expected ingestion profile may also be generated using an estimate for a degree of leakage at a particular wound site, such as a gastrointestinal anastomosis. An electrolytic biosensor, as described herein, may be placed at the point in the gastrointestinal tract for which the expected ingestion profile is determined, for example, in a surgical drain at a wound site. Different expected ingestion profiles may be determined (or estimated) for various degrees of wound leakage in operation 603. The expected ingestion profile may comprise threshold values for conductivity that indicate whether or not the anastomosis is leaking. Then, ingestion profile conductivity data may be collected (operation 604). The collection in operation 604 may involve digital data acquisition and data storage of individual conductivity data points that are correlated in time. The collection in operation 604 may also involve acquiring signal traces comprising a plurality of individual clocked data points. It is noted that in particular embodiments, operations 602-604 may be performed in a different order. For example, when an immediate response to ingestion of a trace fluid is expected, such as with anastomosis sites at the esophagus or the stomach, operation 604 may be initiated prior to ingestion of the trace fluid and may collect baseline conductivity data.

Next in method 600, a decision may be made whether the ingestion profile is complete (operation 606). The completion of the ingestion profile may represent a time period corresponding to the passing of the trace fluid through a location of the gastrointestinal tract where the wound is located. In certain instances, the determination in operation 606 is made based on a time period associated with an expected ingestion profile. When the result of operation 606 is NO, method 600 may loop back to operation 604. When the result of operation 606 is YES, the data collection in operation 604 may stop and the ingestion profile conductivity data may be analyzed (operation 608). The analysis may determine whether the integrity of the wound for which method 600 was performed is intact. The analysis may also determine whether a leak in the wound exists, and optionally, a degree or an extent of the leak. The analysis may involve comparing the ingestion profile conductivity data with reference conductivity data, such as the expected ingestion profile. Other reference conductivity data, such as previously stored ingestion profile conductivity data, may also be used. In some embodiments, reference conductivity data, either for a particular patient or in generalized form, may be retrieved from application server 426 (see FIG. 4). A leak may be detected by an increase in conductivity of the drain effluent that is correlated with expected expulsion of the trace fluid at the wound location. The integrity of the wound may be determined by an absence of the increase in conductivity in the drain effluent associated with the trace fluid. An increase in the conductivity may be determined by comparison with threshold values, which may be pre-determined or may be adaptively or relatively determined. Then, notification of the analysis results may be sent (operation 610). The notification in operation 610 may be sent to wireless user device 420 and/or application server 426 (see FIG. 4). In certain embodiments, application server 426 may be configured to generate an alarm or other message to health care staff in response to analysis results indicating wound leakage and/or in response to a degree of wound leakage. Finally in method 600, ingestion profile conductivity data may be stored (operation 612) for reference.

While the subject of this specification has been described in connection with one or more exemplary embodiments, it is not intended to limit the claims to the particular forms set forth. On the contrary, the appended claims are intended to cover such alternatives, modifications and equivalents as may be included within their spirit and scope.

What is claimed is:

1. An electrolytic biosensor device, comprising:
a processor configured to access memory media; and
a wireless interface,
wherein the memory media store processor instructions executable by the processor to:
balance a bridge circuit that includes a probe installed in a surgical drain at an intracorporeal wound site;
acquire conductivity values from the bridge circuit corresponding to conductivity of a drain effluent in contact with the probe when flowing through the surgical drain;
store the conductivity values; and
transmit the conductivity values via the wireless interface.

2. The electrolytic biosensor device of claim 1, wherein the electrolytic biosensor device is incorporated into the surgical drain.

3. The electrolytic biosensor device of claim 1, further comprising:
the bridge circuit;
a sensor interface coupling the bridge circuit to the probe; and
a power source configured to drive the sensor interface.

4. The electrolytic biosensor device of claim 3, further comprising:
a power control module configured to switch connections to the sensor interface.

5. The electrolytic biosensor device of claim 1, wherein the processor instructions executable to acquire the conductivity values include processor instructions executable to:
perform digital signal processing on output signals from the bridge circuit.

6. The electrolytic biosensor device of claim 1, including processor instructions executable to:
based on the conductivity values, determine wound integrity; and
transmit an indication of the wound integrity via the wireless interface.

7. A method of detecting wound integrity using an electrolytic biosensor, comprising:
determining an expected ingestion profile for a patient ingesting a trace fluid, including estimating a conductivity change over time at a gastrointestinal wound site of the patient;
responsive to receiving an indication of the ingesting of the trace fluid, collecting ingestion profile conductivity data using an electrolytic probe at the gastrointestinal wound site of the patient, wherein the electrolytic probe is installed in a surgical drain and is configured to measure conductivity of an effluent fluid flowing from the gastrointestinal wound site through the surgical drain; and
analyzing the ingestion profile conductivity data to determine fluid integrity of the gastrointestinal wound site, including comparing the ingestion profile conductivity data with the expected ingestion profile.

8. The method of claim 7, wherein the trace fluid has a substantially higher conductivity than a nominal conductivity value of the effluent fluid, and wherein the conductivity change is a substantial increase in conductivity.

9. The method of claim 7, wherein the determining the expected ingestion profile includes:
using a physiological model describing specific attributes of the patient; and
estimating a degree of leakage at the gastrointestinal wound site.

10. The method of claim 7, wherein collecting ingestion profile conductivity data includes storing ingestion profile conductivity data, and wherein analyzing the ingestion profile conductivity data includes comparing new ingestion profile conductivity data to previously stored ingestion profile conductivity data.

11. The method of claim 7, further comprising:
wirelessly transmitting an indication of the fluid integrity of the gastrointestinal wound site to a wireless user device.

12. The method of claim 7, further comprising:
transmitting an indication of the fluid integrity of the gastrointestinal wound site to a server via a network.

13. The method of claim 7, wherein analyzing the ingestion profile conductivity data to determine fluid integrity of the gastrointestinal wound site includes:
determining a degree of leakage at the gastrointestinal wound site.

14. The method of claim 13, further comprising:
based on the degree of leakage, generating an alarm associated with the patient.

15. A non-transitory computer readable memory media storing processor instructions executable by a processor to operate an electrolytic biosensor device, the processor instructions including instructions executable to:
balance a bridge circuit that includes an electrolytic probe installed in a surgical drain at an intracorporeal wound site of a patient, wherein the electrolytic probe includes a conductivity cell for measuring conductivity;
acquire conductivity data from the bridge circuit corresponding to conductivity of a drain effluent flowing through the conductivity cell;
determine fluid integrity of the intracorporeal wound site by comparing the conductivity data with previously-stored reference conductivity data; and
transmit an indication of the fluid integrity of the intracorporeal wound site.

16. The memory media of claim 15, wherein the processor instructions executable to acquire the conductivity data include processor instructions executable to:
control a sensor interface to the conductivity cell; and
process an output signal from the conductivity cell to generate the conductivity data.

17. The memory media of claim 15, including processor instructions executable to:
based on the indication of the fluid integrity of the intracorporeal wound site, generate an alarm associated with the patient.

18. The memory media of claim 15, wherein the processor instructions executable to transmit an indication of the fluid integrity include processor instructions executable to:
send a wireless message to a wireless user device.

19. The memory media of claim 15, including processor instructions executable to:
transmit the conductivity data for the patient to a server.

20. The memory media of claim 15, wherein the processor instructions executable to compare the conductivity data with previously-stored reference conductivity data include processor instructions executable to:
retrieve reference conductivity data for the patient.
